(19) United States
(12) Patent Application Publication
(10) Pub. No.: US 2024/0051927 A1
(43) Pub. Date: Feb. 15, 2024

(54) FLOW SYNTHESIS OF RDX

(71) Applicant: BAE SYSTEMS plc, London (GB)

(72) Inventors: Andy Oden Burn, USK Monmouthshire (GB); Matthew Paul Didsbury, USK Monmouthshire (GB); Stuart Kennedy, Aylesbury Buckinghamshire (GB); Nicola Kennedy, Aylesbury Buckinghamshire (GB); Ian Ewart Murray Paterson, Aylesbury Buckinghamshire (GB); Daniel Jubb, Aylesbury Buckinghamshire (GB)

(73) Assignee: BAE SYSTEMS plc, London (GB)

(21) Appl. No.: 18/256,548

(22) PCT Filed: Dec. 1, 2021
(86) PCT No.: PCT/GB2021/053131

§ 371 Date: Jun. 8, 2023

(30) Foreign Application Priority Data
Dec. 9, 2020 (GB) 2019393.4

(51) Int. Cl. C07D 251/06; C06B 25/34

(52) U.S. Cl. CPC C07D 251/06; C06B 25/34

(57) ABSTRACT

The invention relates to a method for the flow synthesis manufacture of RDX, comprising the steps of preparing input flow reagent A, comprising hexamine dissolved in nitric acid with a concentration less than 92%, preparing input flow reagent B comprising 99% concentration nitric acid, causing the input flow reagents A and B to enter a flow reactor at a flow rate, so as to cause a total nitric acid concentration of greater than 93%, in said flow reactor, cooling the reaction chamber to less than 30° C., causing the output mixed flow to be quenched.

The following invention relates to methods of producing explosives from the direct nitration of hexamine by flow synthesis. Particularly to a method of producing RDX.

Before the present invention is described in further detail, it is to be understood that the invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

According to a first aspect of the invention there is provided a method for the flow synthesis manufacture of RDX, comprising the steps of
i. preparing input flow reagent A, comprising hexamine dissolved in nitric acid with a concentration less than 92%,
ii. preparing input flow reagent B comprising greater than 95% concentration nitric acid,
iii. causing the input flow reagents A and B to enter a flow reactor at a flow rate, so as to cause a total nitric acid concentration of greater than 93%, in said flow reactor,
iv. cooling the reaction chamber to less than 30° C.
v. causing the output mixed flow to be quenched, to cause precipitation of RDX

The use of flow synthesis provides a facile means of preparing RDX at both laboratory R&D scale of ˜100 g, and to provide the ability to add further flow reactors to readily scale up production, without the associated dangers of forming +100 Kgs of RDX explosive in a single reactor vessel. Further, it also avoids the use of hundreds of litres of highly concentrated acid in a large reactor vessel in a batch process. The use of flow synthesis allows for the continuous removal and safe stowage of final explosive product RDX material from the flow reactor or flow reactors, to avoid the build-up of large quantities of explosive material. This may allow explosive processing buildings to process a greater mass of explosive and/or associated safety distances to be reduced, as the explosive material may be distributed to safe areas, away from the flow reactor, as it is synthesised.

The hexamine may be added to the input flow reagent A nitric acid in any wt % up to and including a near saturated solution. The higher the concentration of hexamine in the Input flow reagent A, the more efficient the process. It is highly preferable to dissolve the hexamine in the nitric acid, as short a time as possible before flowing into the reactor, to reduce the likelihood of the nitration reaction starting.

The hexamine may be dissolved in nitric acid with a concentration in the range of from 70% to 92%, more preferably from 88% to 92%, the use of other solvents to aid dissolving the hexamine, may be added.

Preferably input flow reagent A contains only hexamine and nitric acid with a concentration in the range of less and 92%.

The input flow reagent B may comprise 99% concentration nitric acid to ensure the total nitric acid concentration in the flow reactor is at least 92% nitric acid concentration, more preferably input flow reagent B contains only 99% concentration nitric acid.

The use of nitric acid at a concentration below that at which nitration can occur, allows the hexamine starting material to be dissolved, without the nitration reaction starting. This prevents product from precipitating out before it is flowed into the flow reactor, and may prevent blockage of the flow reactor and associated mixing chambers. Further, the use of high percentage concentrations as the dissolving agent for hexamine permits the reaction in the flow reactor to be quickly brought up to the required total nitric acid concentration for nitration to occur. This avoids the issue of having a diluted concentration of nitric acid concentration in the flow reactor, therefore the input flow reagent B only needs to be a slightly higher concentration of nitric acid, to ensure that the desired range of total nitric acid concentration of greater than 92% is achieved in the flow reactor.

To assist in achieving the desirable concentration of nitric acid to start nitration of hexamine, after step ii, the input flow reagents A and B may be premixed in a mixing chamber before entering the flow reactor.

It has been found that in step iii) the total nitric acid concentration may be in the range of 90-99% in said flow reactor, more preferably in the range of 93% to 95% nitric acid concentration.

The total nitric acid concentration when input flow reagent A and input flow reagent B contain only nitric acid as the acid and the sole nitration agent, the concentration must be sufficient for nitration to occur, such as for example greater than 92% concentration.

The flow rate of input flow reagent A may be selected from any suitable flow rate with input flow reagent B, to provide a total nitric acid concertation capable of causing nitration of hexamine, such as for example in the range of greater than 92%. The actual flow rate of input flow reagent A may be μL through to millilitres to litres, depending on the capacity of the flow cell.

The flow rate of input flow reagent B may be selected from any suitable flow rate with input flow reagent A to provide a total nitric acid concertation capable of causing nitration of hexamine, such as for example in the range of greater than 92% concentration. The actual flow rate of input flow reagent A may be μL through to millilitres to litres, depending on the capacity of the flow cell.

The ratio of the flow rate Input flow reagent A to Input flow reagent B (A:B) may be B>A, preferably the ratio is greater than 1:3 (A:B), more preferably in the range of (1:4) to (1:10), to ensure the nitric acid total concentration is greater than 92% in the flow reactor. The use of higher concentrations of acid in Input flow reagent B, allows the volume/flow rate of Input flow reagent B to be reduced, ie a lower ratio, which may lead to reducing the quantity of nitric acid being used. This may be caused by using other strong acids, such as for example oleum.

The temperature in the flow reactor needs to be controlled to prevent a highly exothermic reaction from occurring, preferably the temperature is caused to be less than 30° C., preferably between 20° C. to 30° C., more preferably between 22° C. to 27° C., most preferably at 24° C. The temperature is monitored by water circulators. The flow reactor may be cooled by any suitable means such as for example water circulator or electric coolers.

The reaction in step v, the output mixed flow is quenched, to stop the reaction and to cause precipitation of the RDX product. The output flow may be transferred in to a large volume of quench medium or mixed in a mixing chamber.

Preferably the output mixed flow, which comprises the RDX dissolved in the nitric acid, is mixed with the quench medium via an SOR mixer at the end of the flow reactor. The quench medium may have a pH 7 or less, and may be selected from an aqueous acidic solution or water. The quenching agent may be cooled to induce crystallisation, preferably less than 20° C., preferably in the region of 10° C. or less.

The RDX precipitate is filtered and collected and then washed in an aqueous solution, preferably the quenching solution may have a pH 7 or less, preferably water.

According to a further aspect of the invention there is provided the use of flow synthesis for providing explosives from hexamine.

According to a further aspect of the invention there is provided apparatus for carrying out the process according to any one of the preceding claims, wherein the apparatus is modified for explosive compatibility.

According to a further aspect of the invention there is provided a method for the flow synthesis manufacture of RDX, comprising the steps of
i. preparing input flow reagent A, comprising hexamine dissolved in nitric acid with a concentration less than 92%,
ii. preparing input flow reagent B comprising a nitration reagent,
iii. causing the input flow reagents A and B to enter a flow reactor at a flow rate, so as to cause nitration of RDX in said flow reactor,
iv. cooling the reaction chamber to less than 30° C.
v. causing the output mixed flow to be quenched to; come into contact with an aqueous solution to allow precipitation of RDX.

The nitration reagent may be selected from at least 70% concentration nitric acid and NaNO₂, or containing only 99% concentration nitric acid.

### Experimental Reagents

99% HNO3 was purchased from Honeywell in a 500 mL quantity. Cat. 84392-500ML, Lot. No. 1345S.
70% HNO3 was purchased from Fisher scientific in a 2.5 L quantity. Code: N/2300/PB17. Lot: 1716505.
Hexamine was purchased from Sigma-Aldrich in a 250 g quantity. Cat. 797979-250G, Lot. No. MKCJ7669.
   Oleum was purchased from Fisher in a 500 mL quantity. Cat. S/9440/PB08, Lot. No. 1689177.
   Flow reactor used: 3222 Labtrix

### EXPERIMENTAL

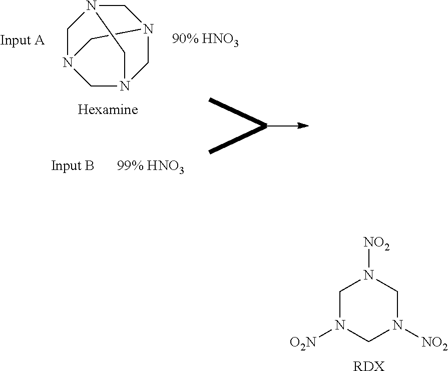

The general reaction is shown above, where the input flow reagent A comprises hexamine dissolved in nitric acid, and input flow reagent B comprises the nitrating agent, which may be higher concentration of nitric acid (than input flow reagent A), and/or a further nitrating agent, such as a metal nitrite, such as NaNO₂. The input flow reagent A and input flow reagent B are caused to react in the flow reactor to furnish the product RDX.

RDX synthesis using a flow reactor poses more challenging design issues than simply pumping solutions from well-known and quantified batch chemistry. This is mainly due to the fact that the starting material hexamine is solid, and RDX can potentially precipitate out of solution during the reaction. Precipitation of the RDX during the transition through the flow reactor can happen as the acid concentration drops and water content increases, thereby leading to potential blockages in the flow reactor, this could lead to catastrophic events, and so the nitric acid concentration in the flow chemistry.

Before starting the experiment the reactor was prepared by flushing the system with methanol followed by water. Both input systems were then filled with 70% HNO₃ which was passed through the reactor in order to fully prime the system.

### Experiment 1

Syringe A: 0.1010 g hexamine in 5 mL 99% HNO₃
Syringe B: 0.1079 g NaNO₂ in 10 mL 70% HNO₃

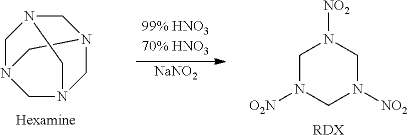

Initial work focused on trying to translate the batch based synthesis directly to the flow reactor. The experiment involved making pre-mixed solutions of hexamine and 99% HNO₃ (solution A) and NaNO₂ in 70% HNO₃ (solution B).

A sample of the initial hexamine stock solution was analysed using ¹H NMR. It was evident from this that the bulk of the reaction had already been completed in the initial hexamine HNO₃ solution before it was passed through the reactor. Therefore, the experimental method was adapted so that the reaction occurs within flow reactor and not the initial solutions.

### Experiment 2—Saturated Hexamine Test

Syringe A: Saturated hexamine in 70% HNO₃ (roughly 1 g in 5 mL).
Syringe B: 99% HNO₃.

It is desirable to use nitric acid as the only nitrating agent, rather than to introduce further reagents, such as for example NaNO₂. Experiment 1 was repeated without the NaNO₂, and using 99% concentration of nitric acid in syringe B to act as the sole nitrating agent.

No precipitate was obtained when the solution from the reactor was collected into water. It can be concluded that the overall acid concentration for the reaction is too low to produce RDX in sufficient quantities therefore focusing on the overall acid concentration of the reaction was investigated.

### Experiment 3 Higher Acid Concentration Test

Syringe A: Saturated hexamine in 90% HNO₃ (roughly 1 g in 5 mL).
Syringe B: 99% HNO₃.

The concentration of the nitric acid in syringe A was increased. The flow rate was set at 1:3 (A:B), however limited product formed. The flow rate of syringe B, 99% HNO₃ feed was increased so that the A:B flow ratio was 1:9. When the sample was collected into water the solution became opaque indicating that RDX had been produced.

### Experiment 4 Addition of Oleum

Syringe A: 0.5 g hexamine dissolved in 2.5 mL 90% HNO₃. Solution cooled during hexamine addition.
Syringe B: 0.95 mL 99% HNO₃+0.05 mL oleum.

A series of experiments were carried out aimed at monitoring the influence of oleum on RDX formation. Experiments produced opaque solutions when collected into water indicative of RDX formation. The ¹H NMR spectrum showed that RDX exists in solution prior to precipitation using water as the quenching agent.

### Experiment 5 Increased Oleum Addition

Syringe A: 0.5 g hexamine dissolved in 2.5 mL 90% HNO₃. Solution cooled during hexamine addition.
Syringe B: 0.9 mL 99% HNO₃+0.1 mL oleum.

The increase of oleum by 100%, led to formation of RDX precipitate when collected onto ice. Part of the solution before mixing with ice was collected into d⁶-DMSO, the ¹H NMR spectrum indicated the formation of RDX.

The use of further acids such as oleum, helps to keep that acid concentration in the reactor at a high level, and may assist in dehydration of the reaction. The use of nitration species such as NaNO₂, can allow the use of lower total nitric acid concentrations.

It was found that low acidity in the reactor caused RDX to precipitate from the solution. It is essential to monitor the flow reactor paths for solidified product. Further, whilst it is desirable to increase the acidity of the nitric acid that comprises the hexamine, if the concentration is too high product starts to form, before mixing has commenced, again leading to likelihood of RDX product blocking the flow reactor. Preferably the hexamine is dissolved in the nitric acid, before use, and is not stored long term as a stock solution.

1. A method for the flow synthesis manufacture of RDX, the method comprising:
preparing input flow reagent A, comprising hexamine dissolved in nitric acid with a concentration less than 92%;
preparing input flow reagent B, comprising greater than 95% concentration nitric acid;
causing the input flow reagents A and B to enter a reaction chamber of a flow reactor at a flow rate, so as to cause a total nitric acid concentration of greater than 93%, in said flow reactor;
cooling the reaction chamber to less than 30° C.; and
causing an output mixed flow to be quenched, to cause precipitation of RDX.

2. The method according to claim 1, wherein the hexamine is dissolved in nitric acid with a concentration in the range of from 88% to 90%.

3. The method according to claim 1, wherein the hexamine is dissolved in nitric acid to achieve a saturated solution.

4. The method according to claim 1, wherein the input flow reagent B is 99% concentration nitric acid.

5. The method according to claim 1, wherein the total nitric acid concentration is in the range of 90-99%, in said flow reactor.

6. The method according to claim 1, wherein the flow rate ratio of input flow reagent A to input flow reagent B (A:B) is greater than 1:3 (A:B).

7. The method according to claim 1, wherein the temperature in said flow reactor is in the range of from 20° C. to 27° C.

8. The method according to claim 1, wherein the nitric acid in input flow reagent B further comprises oleum or NaNO₂.

9. The method according to claim 1, wherein the quench is caused by mixing the output mixed flow and a quenching agent.

10. The method according to claim 9, wherein the quenching agent is an aqueous solution, so as to cause precipitation of RDX.

11. The method according to claim 9, wherein the quenching agent is cooled below 10° C.

12. Apparatus for carrying out the process according to claim 1, wherein the apparatus is configured for explosive compatibility.

13. A method comprising the use of flow synthesis for providing explosive material from hexamine.

14. A method for the flow synthesis manufacture of RDX, the method comprising:
preparing input flow reagent A, comprising hexamine dissolved in nitric acid with a concentration less than 92%;
preparing input flow reagent B, comprising a nitration reagent;
causing the input flow reagents A and B to enter a reaction chamber of a flow reactor at a flow rate, so as to cause nitration of RDX in said flow reactor;
cooling the reaction chamber to less than 30° C.; and
causing an output mixed flow to be quenched, to allow precipitation of RDX.

15. The method according to claim 14, wherein the nitration reagent is selected from at least 70% concentration nitric acid and NaNO₂, or containing only 99% concentration nitric acid.

16. The method according to claim 14, wherein causing the output mixed flow to be quenched includes causing the output mixed flow to come into contact with an aqueous solution.

17. The method according to claim 14, wherein the nitration reagent comprises at least 70% concentration nitric acid and NaNO₂.

18. The method according to claim 14, wherein the nitration reagent comprises 99% concentration nitric acid.

19. The method according to claim 1, wherein the total nitric acid concentration is in the range of 93% to 95%, in said flow reactor.

20. The method according to claim 1, wherein the total nitric acid concentration is in the range of 93% to 99%, in said flow reactor.
